(19) United States
(12) Patent Application Publication
(10) Pub. No.: US 2023/0277440 A1
(43) Pub. Date: Sep. 7, 2023

(54) EFFERVESCENT GEL DRY POWDER FORMULATION AND PREPARATION METHOD THEREOF

(71) Applicant: NINGBO FOCUS BIOMEDICAL TECHNOLOGY CO., LTD., Ningbo, Zhejiang (CN)

(72) Inventors: Qiuye ZHANG, Ningbo, Zhejiang (CN); Yansong WANG, Ningbo, Zhejiang (CN)

(21) Appl. No.: 18/007,043

(22) PCT Filed: Aug. 3, 2021
(86) PCT No.: PCT/CN2021/110337

§ 371 Date: Jan. 26, 2023

(30) Foreign Application Priority Data
Sep. 11, 2020 (CN) 202010956496.5

(51) Int. Cl. A61K 9/46; A23L 2/40; A61K 31/545; A61K 47/02; A61K 47/10; A61K 47/32

(52) U.S. Cl. CPC A61K 9/0007; A23L 2/40; A61K 31/545; A61K 47/02; A61K 47/10; A61K 47/32

(57) ABSTRACT

Effervescent gel EG agent is a new oral formulation platform. It includes a fast-expanding gel agent, a foam-forming agent, an active ingredient, and an auxiliary ingredient. Effervescent gel is mixed with appropriate amount of aqueous agent or aqueous solution, which can swell rapidly (generally less than 30 seconds) and form a uniform multi-bubble hydrogel, which has the characteristics of fast gel formation, convenient preparation, uniform gel, smooth taste and adjustable viscosity, especially for children, the elderly and patients with swallowing reflex dysfunction. Oral effervescent gels are convenient, safe and have excellent taste, and significantly increase the compliance of pediatric patients, and can be used in the fields of pharmaceuticals, food and health products.

### TECHNICAL FIELD

The invention relates to an oral preparation for medicine, food and healthcare product, specifically to an effervescent gel dry powder preparation and preparation method thereof.

### BACKGROUND

Oral preparations generally include tablets, capsules, suspensions, granules, solutions, syrups and gels. For infants and some patients with special swallowing reflex dysfunction, due to their age, physical or disease reasons, their swallowing reflex is impaired, and during the swallowing process, it is easy to aspirate liquid or solid oral substances into the trachea, resulting in aspiration pneumonia or asphyxia or even death. Therefore, the most suitable oral dosage form for him/her is a gel with a certain viscosity and adjustable viscosity. A certain viscosity can make the gel adhere to the mucous membrane of the mouth and pharynx, so that it can only enter the esophagus through swallowing and prevent accidental aspiration into the trachea; a certain consistency can make the gel form a larger block and reduce its mobility, which is easy to swallow and can prevent the oral substance from flowing into the trachea due to the gravity of body position.

according to the different severity of the patient dysfunction, different eating habits, the different patient are met by the gel remove different viscosities. At present, there are few gels in the market, the viscosity is fixed, it is not adjustable, in the preparation process, it needs to add preservative, and it needs to be stored at low temperature.

Due to the defect of the current market gel, it can not meet the requirement of infant and some patient reflex dysfunction, and the current gel is not freshly prepared, the taste is bad, and it is not easy to obtain wide application.

### CONTENTS OF THE INVENTION

The technical problem to be solved by the invention is to provide a new type oral effervescent gel with adjustable viscosity. The invention can meet the requirements of infants and some patient oral medicine and food with function of swallowing and reflecting functional disorder.

In order to realize the technical effect, the present invention provides an effervescent gel dry powder preparation, the effervescent gel dry powder preparation comprises one of pharmaceutical ingredient or active ingredient, gel particle, agent and auxiliary filler; the said gel the weight percentage of the quick-swelling gel in the effervescent gel dry powder preparation is 5% to 15%; and the weight percentage of the effervescent agent gel is not less than ⅕ of the weight percentage of the gel dry powder preparation of the effervescent gel.

In one embodiment, the pharmaceutical ingredient is cephalosporin medicine, macrolide medicine, ribavirin, oseltamivir, fumaric acid, ibuprofen, acetaminophen, vitamin medicine, dextromethorphan hydrobromide or Chinese patent medicine particles.

In one embodiment, the pharmaceutical ingredient is 1% to 50% of the weight percentage of the effervescent gel dry powder preparation.

In one embodiment, the active ingredient is Lactobacillus acidophilus, AKK, Bifidobacterium, Lactobacillus rhamnosus, Clostridium butyricum, Saccharomyces cerevisiae, Enterococcus, Bacillus licheniformis or Bacillus cereus.

In one embodiment, the gel particle card is BmM971P uniformly adhered to the polyethylene glycol 6000 wax particle surface to form a compact particulate matter

In one embodiment, the bubble agent sodium bicarbonate, the bubble is agent % to 5% of the weight percentage of the effervescent gel dry powder preparation.

In one embodiment, the auxiliary filler comprises flavouring agent, taste agent and agent, the flavouring agent accounts for 40% to 90% of the weight percentage of the effervescent gel dry powder preparation; the taste masking agent accounts for 0% to 10% of the weight percentage of the effervescent gel dry powder preparation; the weight percentage of the coloring is agent % to 6% of the effervescent gel dry powder preparation.

Another aspect of the present invention is to provide an effervescent gel dry powder pre-adjusting agent, the effervescent gel dry powder pre-adjusting agent for mixing with other granular preparation to prepare effervescent gel preparation, the effervescent gel dry powder pre-adjusting agent comprises gel agent and the weight percentage of the effervescing agent gel not less than ⅕ of the weight percentage of the gel dry powder pre-adjusting agent of the effervescent tablet gel the particle card of the gel the Bum 971P, which is uniformly adhered on the surface of the wax particle of the polyethylene glycol 6000 to form a compact particulate matter The bubble-making agent is sodium bicarbonate the gel particle diameter is particulate matter-250 microns, and because of the large amount of carboxyl of the carbomer or the carbophil; It reacts with sodium bicarbonate in the sodium bicarbonate solution to produce water and carbon dioxide gas, at the same time, the carbomer will become thick to form gel in the alkaline solution. Therefore, the quick-expansion gel of the present invention and the bubble agent mixture can quickly form bubbles gel mixing with water, at the same time, the drug or active substance is wrapped.

The inventive point of the present invention is in the following aspects:
1. The invention Claims a method for using the method for matching the gel the particle with the foam agent produce the gel, so as to generate a new oral preparation type-effervescent gel, improving the taste and smoothness of the gel technology.
2. The present invention further determines the use content of the gel particle by experiment, so as to obtain the best taste and gel of the preparation. At the same time, the use agent between the gel

### DESCRIPTION OF PICTURES

The drawings are used to provide a further understanding of the present invention and constitute a part of the specification, and the embodiments of the present invention are used to explain the present invention, and do not constitute the limit to the present invention. In the drawings:

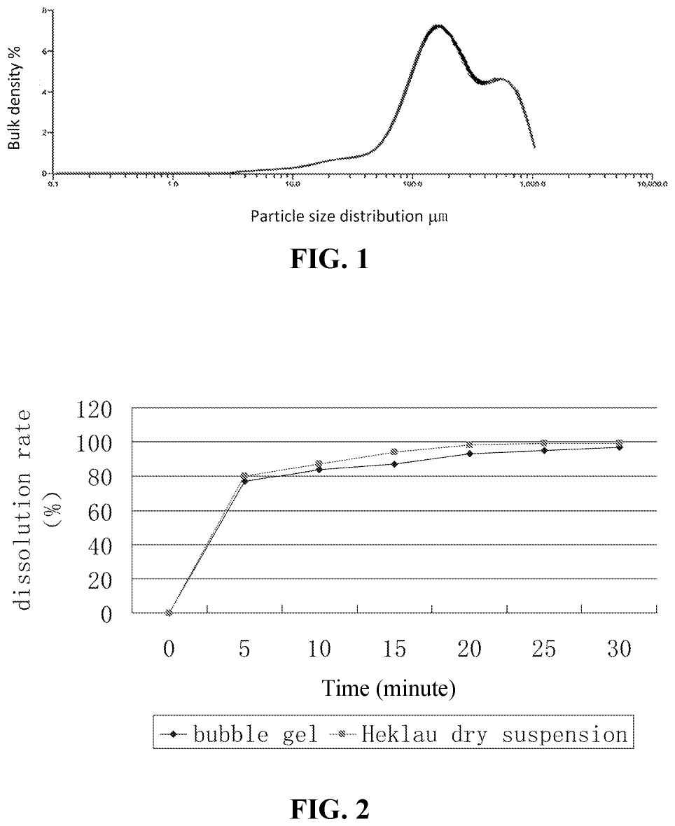

FIG. 1 is a particle size distribution diagram of the quick-expansion gel used in the present invention.

FIG. 2 is a schematic diagram of dissolution rate of the bubble gel by the present invention on the cefaclor component in the existing technology of schig dry suspension preparation on the cefaclor component.

### SPECIFIC IMPLEMENTATION EXAMPLES

The embodiments of the present invention will be further illustrated in conjunction with the accompanying drawings.

### Embodiment 1

The above is only the specific embodiments of the present invention, the protection range of the invention is not limited to this, any familiar with the technical personnel of the invention in the technical specification of the invention, the modification or replacement of the invention should be in the protection range of the invention.

In this embodiment, firstly preparing fast swelling gel particles, the preparation process of the fast swelling gel particles is as follows:

placing the 500 g carbomer 971P in a wet granulating machine pot, heating the carbomer 971P by the interlayer of the pot by 95 degrees water bath. under the stirring paddle of 15 r/min stirring, carbomer 971P gradually heating, when the temperature reaches to 85-90 degrees centigrade, the polyethylene glycol 6000 (PEG6000) particles 320 g slowly into the wet granulating machine pot, then the stirring paddle speed is increased to 30 r/min; keeping the water bath at 95 degrees centigrade for heating after about 2-10 minutes, carbomer uniformly adhered to the polyethylene glycol 6000 wax particle surface to form a compact particle. after granulating, pouring out the material, slightly cooling and sieving by 20-mesh sieve, namely quick-swelling gel particles is prepared. The quick-swelling gel particles have an average particle diameter of about 150 microns. The particle size distribution of the quick-swelling gel particles is shown in FIG. 1.

taking 10 g of the prepared quick-swelling gel, 6 g of pharmaceutical active ingredient, 2 g of bubble agent g of flavouring agent, 3 g of taste masking agent, 1 g of coloring agent fully mixing to prepare 100 g effervescent gel dry powder preparation of this embodiment. The pharmaceutical active ingredient used in this embodiment, the compound of the specific use of the foam agent flavoring agent, taste masking and agent agent as shown in Table 1 below.

TABLE 1

| Component | Specific compounds |
|---|---|
| Pharmaceutical active ingredient | cefaclor |
| A bubble-making agent | Sodium bicarbonate |
| A flavouring agent | Sucrose powder |
| A taste masking agent | Fruit flavor essence |
| Coloring agent | sunset yellow |

### Embodiment 2

In this embodiment, firstly preparing quick-swelling gel particles, the preparation process of the quick-swelling gel particles is completely consistent with the preparation process of the quick-swelling gel particles recorded in Example 1.

taking 10 g of the prepared quick-swelling gel, 10 g of pharmaceutical active ingredient, 3 g of foam agent 73 g of flavouring agent, 3 g of taste masking agent, 1 g of coloring agent fully mixing to prepare 100 g effervescent gel dry powder preparation of this embodiment. The pharmaceutical active ingredient used in this embodiment, the compound of the specific use of the foam agent flavoring agent, taste masking and agent agent as shown in Table 2 below.

TABLE 2

| Component | Specific compounds |
|---|---|
| Pharmaceutical active ingredient | cefaclor |
| A bubble-making agent | Sodium bicarbonate |
| A flavouring agent | Xylitol |
| A taste masking agent | Fruit flavor essence |
| Coloring agent | sunset yellow |

### Embodiment 3

In this embodiment, firstly preparing quick-swelling gel particles, the preparation process of the quick-swelling gel particles is completely consistent with the preparation process of the quick-swelling gel particles recorded in Example 1.

Selecting the above preparation of quick-expansion gel particle 12 g, medicine active ingredient 8 g, bubble agent g of flavouring agent, 72 g of taste masking agent, 3 g of colouring agent g. fully mixing to prepare 100 g effervescent gel dry powder preparation of this embodiment. The pharmaceutical active ingredient used in this embodiment, the compound of the specific use of the foam agent flavoring agent, taste masking and agent, agent shown in Table 3 below.

TABLE 3

| Component | Specific compounds |
|---|---|
| Pharmaceutical active ingredient | cefaclor |
| A bubble-making agent | Sodium bicarbonate |
| A flavouring agent | Xylitol |
| A taste masking agent | Fruit flavor essence |
| Coloring agent | sunset yellow |

### Embodiment 4

In this embodiment, firstly preparing quick-swelling gel particles, the preparation process of the quick-swelling gel particles is completely consistent with the preparation process of the quick-swelling gel particles recorded in Example 1.

taking 15 g of the prepared quick-expansion gel, 12 g of pharmaceutical active ingredient, 4 g of brew agent 58 g of flavouring agent, 8 g of taste masking agent, 1 g of coloring agent fully mixing to prepare 100 g effervescent gel dry powder preparation of this embodiment. The pharmaceutical active ingredient used in this embodiment, the compound of the specific use of the foam agent flavoring agent, taste masking and agent, agent shown in Table 4 below.

TABLE 4

| Component | Specific compounds |
|---|---|
| Pharmaceutical active ingredient | cefaclor |
| A bubble-making agent | Sodium bicarbonate |
| A flavouring agent | Sucrose powder |
| A taste masking agent | Fruit flavor essence |
| Coloring agent | sunset yellow |

### Comparative Example 1

In this embodiment, firstly preparing quick-swelling gel particles, the preparation process of the quick-swelling gel particles is completely consistent with the preparation process of the quick-swelling gel particles recorded in Example 1.

Selecting the above preparation of quick-swelling gel particle 4 g, medicine active ingredient 10 g, bubble agent g of flavouring agent, 80 g of taste masking agent, 3 g of colouring, agent g. fully mixing to prepare 100 g effervescent gel dry powder preparation of this embodiment. The pharmaceutical active ingredient used in this embodiment, the compound of the specific use of the foam agent flavoring agent, taste masking and agent, agent shown in Table 5 below.

TABLE 5

| Component | Specific compounds |
|---|---|
| Pharmaceutical active ingredient | cefaclor |
| A bubble-making agent | Sodium bicarbonate |
| A flavouring agent | Sucrose powder |
| A taste masking agent | Fruit flavor essence |
| Coloring agent | sunset yellow |

### Comparative Example 2

In this embodiment, firstly preparing quick-swelling gel particles, the preparation process of the quick-swelling gel particles is completely consistent with the preparation process of the quick-swelling gel particles recorded in Example 1.

taking 18 g of the prepared quick-swelling gel, 6 g of the medicine active ingredient, 8 g of the bubble, agent g of the flavouring agent, 3 g of the taste masking agent and 1 g of the colouring agent. fully mixing to prepare 100 g effervescent gel dry powder preparation of this embodiment. The pharmaceutical active ingredient used in this embodiment, the compound of the specific use of the foam agent flavoring agent, taste masking and agent, agent shown in Table 5 below.

TABLE 5

| Component | Specific compounds |
|---|---|
| Pharmaceutical active ingredient | cefaclor |
| A bubble-making agent | Sodium bicarbonate |
| A flavouring agent | Sucrose powder |
| A taste masking agent | Fruit flavor essence |
| Coloring agent | sunset yellow |

### Comparative Example 3

In this embodiment, firstly preparing quick-swelling gel particles, the preparation process of the quick-swelling gel particles is completely consistent with the preparation process of the quick-swelling gel particles recorded in Example 1.

Selecting the above preparation of quick-swelling gel 13 g, medicine active ingredient 6 g, bubble agent 2 g, flavouring agent 75 g, taste masking agent 3 g, coloring agent g. fully mixing to prepare 100 g effervescent gel dry powder preparation of this embodiment. The pharmaceutical active ingredient used in this embodiment, the compound of the specific use of the foam agent flavoring agent, taste masking and agent, agent shown in Table 5 below.

TABLE 5

| Component | Specific compounds |
|---|---|
| Pharmaceutical active ingredient | cefaclor |
| A bubble-making agent | Sodium bicarbonate |
| A flavouring agent | Sucrose powder |
| A taste masking agent | Fruit flavor essence |
| Coloring agent | sunset yellow |

preparing 10 g agent the prepared effervescent gel dry powder into a clean container, mixing uniformly, adding 25 ml of water, lightly stirring for about 10-20 seconds with small soup spoon, mixing the effervescent gel dry powder preparation with water, quickly expanding, forming uniform effervescent gel the embodiment 1-4 and Comparative Example 1-3 formed by the mouthfeel gel is shown in Table 6.

TABLE 6

| Example | smooth degree | viscosity |
|---|---|---|
| Embodiment 1 | preferably | preferably |
| Embodiment 2 | preferably | preferably |
| Embodiment 3 | preferably | preferably |
| Embodiment 4 | preferably | preferably |
| Comparative Example 1 | preferably | dilute |
| Comparative Example 2 | preferably | too thick |
| Comparative Example 3 | flocculent block | preferably |

Patients with swallowing disorders are easy to cause choked cough when food is diluted; When the food is thick, it is easy to cause dysphagia. At the same time, in order to make the medicine or active substance fully dissolve in the water, the dosage of the used water is stored in the lowest limit dosage. According to Table 6, the weight percentage content of the quick-expansion gel of the invention is set between 5% to 15%, so as to ensure the prepared effervescent gel dry powder preparation obtained after mixing with gel has a proper viscosity, so that the swallowing disorder patient can be more easily for oral action. at the same time, in order to avoid forming flocculent agglomeration in the gel, the weight percentage of the foam agent greater than ⅕ of the weight percentage of the effervescent gel dry powder, so as to ensure the effervescent gel dry powder preparation after mixing with water to form uniform, gel flocculent agglomeration. The dissolution rate comparison result of the bubble gel the present market and the present market is as shown in FIG. 2, it can be seen that the bubble gel the invention is released on the drug in vitro and similar to the Hilg dry suspension, 30 minutes can reach substantially complete release, there is no significant difference.

### Embodiment 5

The embodiment is to make a bubble gel powder pre-adjusting agent, in this embodiment, firstly preparing quick-swelling gel particles, the preparation process of the quick-swelling gel particles is completely consistent with the preparation process of the quick-swelling gel particles recorded in Example 1.

taking 100 g of the prepared quick-expansion gel and fully mixing with 20 g of sodium bicarbonate to prepare 120 g of the bubble-gel dry powder pre-adjusting agent in this embodiment. taking the 2 g of bubble gel powder pre-adjusting agent in clean container, then adding medicine after mixing uniformly, adding 25 ml of water, lightly stirring for about 15-20 seconds with small soup spoon, effervescent gel dry powder preparation after mixing with water, quickly expanding to form uniform effervescent gel The obtained gel mixing has a suitable viscosity, so that the swallowing disorder patient can be more easily for oral action.

As can be seen by the embodiment of the present invention provides a bubble gel dry powder preparation or gel dry powder pre-agent can realize fast forming suitable for making the patient with dysphagia patient easy to oral, gel invention point of the invention is mainly in two aspects, the first, The invention Claims a method for producing foam-containing gel by using the method of matching the gel containing particles with the foam-making agent, the invention uses the Carbomer or polycarbophil non-large carboxyl, the sodium bicarbonate solution reacts with sodium bicarbonate to produce water and carbon dioxide gas, so as to improve the taste and gel of the preparation in the existing technology. the second, the invention further determines the use content of the gel particle and the use ratio gel the particle and the bubble agent so as to obtain the best taste and smooth gel of the preparation.

1. An effervescent gel dry powder agent, wherein the effervescent gel dry powder agent comprises one of pharmaceutical ingredient or active ingredient, fast-expanding gel particle, foaming agent and auxiliary ingredients; the fast-expanding gel particles are composed of carbomer or polycarbophil non-uniformly adhered to the surface of polyethylene glycol particles; the weight percentage of the fast-expanding gel particles in the effervescent gel dry powder agent is 5% to 15%; and the weight percentage of the foaming agent in the effervescent gel dry powder agent is not less than ⅕ of the weight percentage of the fast-expanding gel particles in the effervescent gel dry powder agent.

2. The effervescent gel dry powder preparation according to claim 1, wherein the pharmaceutical ingredient is cephalosporin medicine, macrolide medicine, ribavirin, oseltamivir, ferrous fumarate, ibuprofen, acetaminophen, vitamin medicine, dextromethorphan hydrobromide or Chinese patent medicine particles.

3. The effervescent gel dry powder preparation according to claim 1, wherein the active ingredient is Lactobacillus plantarum, Lactobacillus acidophilus, AKK, Bifidobacterium, Lactobacillus rhamnosus, Clostridium butyricum, Saccharomyces cerevisiae, Enterococcus, Bacillus licheniformis or Bacillus cereus.

4. The effervescent gel dry powder preparation according to claim 1, wherein the weight percentage of the pharmaceutical ingredient is 1% to 50% of the effervescent gel dry powder preparation.

5. The effervescent gel dry powder preparation according to claim 1, wherein the fast-expanding gel particles is carbomer 971P, carbomer 974P, carbomer 934P or polycarbopol non-uniformly adhered to the polyethylene glycol wax particle surface, forming a relatively dense particulate matter the polyethylene glycol is polyethylene glycol 4000, polyethylene glycol 6000, polyethylene glycol 8000, polyethylene glycol 10000 or polyethylene glycol 12000.

6. The effervescent gel dry powder preparation according to claim 1, wherein the agent is sodium bicarbonate, the foam is agent % to 5% of the weight percentage of the effervescent gel dry powder preparation.

7. The effervescent gel dry powder preparation according to claim 1, wherein the auxiliary filler comprises flavouring agent, taste agent and agent, the flavouring agent accounts for 40% to 90% of the weight percentage of the effervescent gel dry powder preparation; the taste masking agent accounts for 0% to 10% of the weight percentage of the effervescent gel dry powder preparation; the weight percentage of the coloring is agent % to 6% of the effervescent gel dry powder preparation.

8. An effervescent gel dry powder pre-adjusting agent, wherein the effervescent gel dry powder pre-adjusting agent is used for mixing with other granular preparation to prepare effervescent gel preparation, the effervescent gel dry powder pre-adjusting agent comprises fast-expanding gel particles and forming agent, and the weight percentage of the foaming agent in the effervescent gel dry powder agent is not less than ⅕ of the weight percentage of the fast-expanding gel particles in the effervescent gel dry powder agent; the fast-expanding gel particles are carbomer 971P uniformly adhered to the surface of polyethylene glycol 6000 wax particles to form relatively dense particles; The bubble-making agent is sodium bicarbonate or potassium bicarbonate.
